(19) United States
(12) Patent Application Publication
(10) Pub. No.: US 2022/0355077 A1
(43) Pub. Date: Nov. 10, 2022

(54) TUBE SECUREMENT SYSTEM

(71) Applicant: 3M INNOVATIVE PROPERTIES COMPANY, SAINT PAUL, MN (US)

(72) Inventors: JAMES M. SIERACKI, PLYMOUTH, MN (US); KRYSTAL J. SCHEIBEL, MINNEAPOLIS, MN (US)

(73) Assignee: 3M INNOVATIVE PROPERTIES COMPANY, SAINT PAUL, MN (US)

(21) Appl. No.: 17/636,913

(22) PCT Filed: Sep. 22, 2020
(86) PCT No.: PCT/IB2020/058843

§ 371 Date: Feb. 21, 2022

Related U.S. Application Data
(60) Provisional application No. 62/913,796, filed on Oct. 11, 2019.

(51) Int. Cl. A61M 25/02; A61F 13/02

(52) U.S. Cl. CPC A61M 25/02; A61F 13/023; A61M 2025/0266; A61F 2013/00412

(57) ABSTRACT

The disclosed tape system comprises a first tape and second tape each with an adhesive that interact to secure a device such as tubing. The first tape and second tape each have a longitudinally extending midsection and laterally extending legs. The first tape overlaps the device, while the second tape is under the device, but with the legs of the second tape overlapping the legs of the first tape. This overlapping connection of the first tape and second tape strongly secures the tubing.

### TECHNICAL FIELD

The present disclosure generally relates to a medical article securement systems and methods for securing medical articles to the body of a patient, and particularly, for securing various catheter system, tubes, or other elongated devices to the body of a patient.

### BACKGROUND

Tubes and catheters are inserted through a patient's skin to either introduce fluids to the patient or to remove fluids from the patient. The tubes must be secured to prevent the tube from slipping out of the patient. Commonly, surgical tape or sutures are used to hold tubing to the patient's skin.

One very common type of tube is an intravenous (IV) catheter. An IV catheter is inserted into the patient's bloodstream. Movement of an IV catheter while the catheter is inserted in a vein is a leading cause of catheter failure. When a catheter moves in a vein, it scrapes and pokes the inner wall of the vein, thereby irritating the vein. Repeated movement of the catheter can cause sufficient irritation of the vein to require that the catheter be removed and then a new catheter to be inserted in a different location along the same vein or in an entirely different vein. Therefore, a need exists for effective securement systems for tubes, such as catheters.

### SUMMARY

The disclosed tape system comprises a first tape and second tape each with an adhesive that interacts to secure a device such as tubing. The first tape and second tape each have a longitudinally extending midsection and laterally extending legs. The first tape overlaps the device, while the second tape is under the device, but with the legs of the second tape overlapping the legs of the first tape. This overlapping connection of the first tape and second tape strongly secures the tubing.

In one embodiment, the tape system includes first and second tapes, each tape having a first major surface comprising an adhesive, and a second major surface opposite the first major surface. The first and second tapes each have a midsection, and first and second legs extending from first and second ends of the midsection, the two legs extending in a direction lateral to the midsection. The first tape is applied over the second tape such that the first lateral direction of the first tape is opposed to the first lateral direction of the second tape. The midsections of the first and second tapes are aligned, but spaced apart from each other.

In a further embodiment, the second tape is applied over the first tape with the first leg of the second tape applied over the second leg of the first tape, and with the second leg of the second tape applied over the first leg of the first tape.

In one embodiment, the tape system includes a dressing overlying a device and a substrate. At least a portion of the tape system can be adhered to the dressing. At least a portion of the tape system can be adhered to the substrate. In some embodiments, the substrate is skin.

In one embodiment, a method for securing a device to a substrate includes applying the tape system to the device, the adhesive at the midsection of the first tape overlying a portion of the device with the first leg and second leg of the first tape adjacent to the device and overlying the substrate, and applying the midsection of the second tape of claim 1 under a portion of the device with the adhesive towards the substrate. The first leg of the second tape is overlying the second leg of the first tape, the second leg of the second tape is overlying the first leg of the first tape, and the device extends between the midsection of the first tape and the midsection of the second tape.

In a further embodiment, the method includes applying a dressing over the device, and under at least a portion of the tape system.

In some embodiments, the substrate is skin.

In one embodiment, a kit is provided that includes: 1) a dressing system and a first tape, the entire first tape overlying the dressing film; and 2) a second tape. The first and second tapes include features of the first and second tapes in embodiments of the tape system.

### BRIEF DESCRIPTION OF DRAWINGS

FIG. 1 is a first embodiment of a tape;

FIG. 2 is a side sectional view of FIG. 1 through line 2-2;

FIG. 3 is an alternate embodiment of a tape;

FIG. 4 is an embodiment of a tape system securing a tube in place;

FIG. 5 is the tape system of FIG. 4 with an underlying dressing;

While the above-identified drawings and figures set forth embodiments of the invention, other embodiments are also contemplated, as noted in the discussion. In all cases, this disclosure presents the invention by way of representation and not limitation. Other modifications and embodiments can be devised by those skilled in the art, which fall within the scope and spirit of this invention. The figures may not be drawn to scale.

### DETAILED DESCRIPTION

Tape System

The disclosed tape system 1 comprises a first tape 100 and second tape 200 each with an adhesive that interact to secure a device such as tubing. The first tape and second tape each have a longitudinally extending midsection and laterally extending legs. The first tape overlaps the device, while the second tape is under the device, but with the legs of the second tape overlapping the legs of the first tape. This overlapping connection of the first tape and second tape strongly secures the tubing.

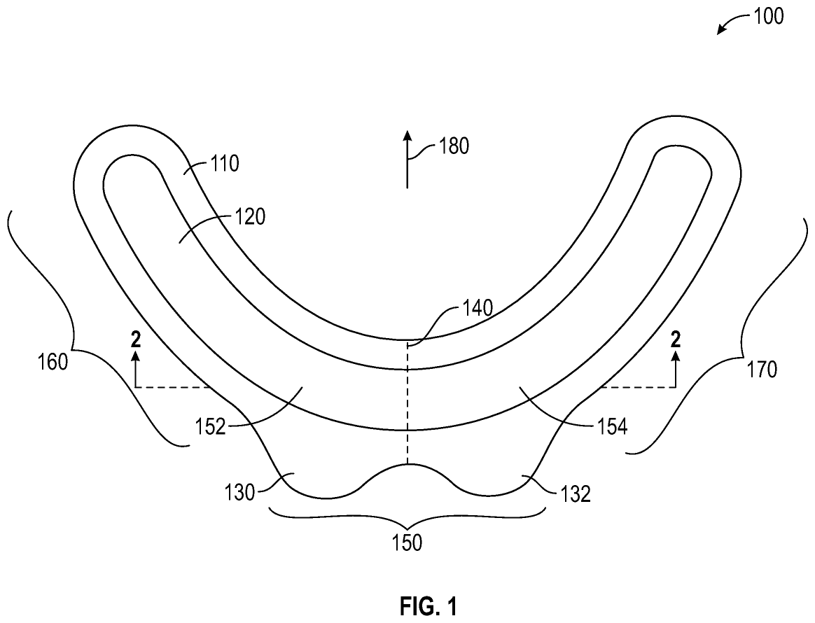

FIG. 1 is an embodiment of the first tape 100 having a backing 110. Backing 110 has a first major surface and a second major surface opposite the first major surface. The first major surface of backing 110 has an adhesive. Tape 100 has midsection 150, the midsection having a first end 152 and a second end 154. A first leg 160 extends away from midsection 150 at first end 152 in a lateral direction 180, and a second leg 170 extends away from midsection 150 at second end 154 in the lateral direction 180. Midsection 150 extends in a longitudinal direction perpendicular to lateral direction 180. In this embodiment, the midsection 150 is curved with the first leg 160 and second leg 170 extending along the same curved shape forming a “C” shaped tape 100. In this embodiment, the tape 100 is symmetrical. Other shapes and configurations of the tape 100 can be used such as being asymmetrical, having wider and narrower portions, legs of differing lengths, straight, curved or a combination for the arrangements of the midsection and legs, so long as the legs extend in the same lateral direction.

Optionally, midsection 150 has tabs 130 and 132 extending away from midsection 150. Midsection 150 optionally includes a perforation set 140 extending laterally across midsection 150. Optional tabs 130 and 132, and optional perforation set 140 can be beneficial in removal of the tape system.

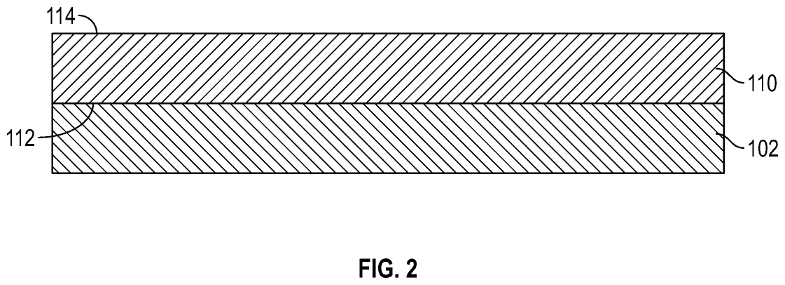

FIG. 2 show a side sectional view of tape 100, showing backing 110 as a layer having a first major surface 112 and a second major surface 114 opposed to first major surface 112. First major surface 112 of backing 110 has a layer of adhesive 102. In some embodiments, the adhesive is a skin-adhesive.

In some embodiments, backing 110 can include multiple layers, including, for example, a reinforcement layer 120 to provide stiffness and/or strength to tape 100 for securing a device, such as a tube, or more specifically a catheter. In some embodiments, reinforcement layer 120 is disposed at second major surface 114. The reinforcement layer may be visually clear or have a color to indicate its presence.

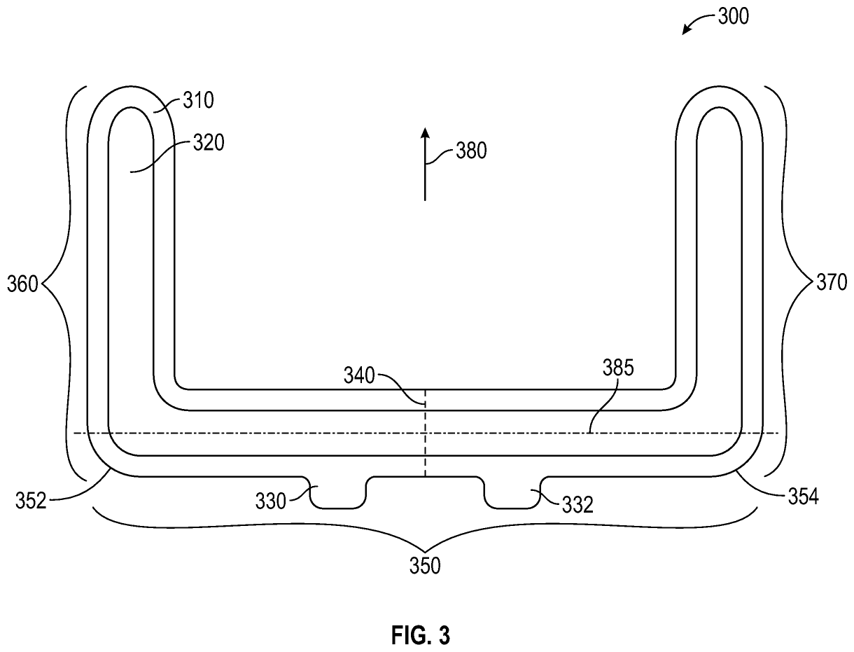

FIG. 3 illustrates an alternate embodiment of a tape 300 having a backing 310. In contrast the “C” shape of tape 100, tape 300 has a more rectilinear shape. Backing 310 has a first major surface and a second major surface opposite the first major surface. The first major surface of backing 310 has an adhesive. Tape 300 has midsection 350 extending in a longitudinal direction along axis 385, the midsection having a first end 352 and a second end 354. A first leg 360 extends away from midsection 350 at first end 352 in a lateral direction 380, and a second leg 370 extends away from midsection 350 at second end 354 in the lateral direction 380. The longitudinal direction is perpendicular to the lateral direction 380. In this embodiment, the tape 300 is symmetrical. Other shape and configurations of the tape 300 can be used such as being asymmetrical, having wider and narrower portions, legs of differing lengths, straight, curved or a combination for the arrangements of the midsection and legs, so long as the legs extend in the same lateral direction.

Optionally, midsection 350 has tabs 330 and 332 extending away from midsection 350. Midsection 350 optionally includes a perforation set 340 extending laterally across midsection 350. Optional tabs 330 and 332, and optional perforation set 340 can be beneficial in removal of the tape system.

In some embodiments, backing 310 can include multiple layers, including, for example, a reinforcement layer 320 to provide stiffness and/or strength to tape 300 for securing a device, such as a tube, or more specifically a catheter. In some embodiments, reinforcement layer 320 is disposed at the second major surface of backing 310.

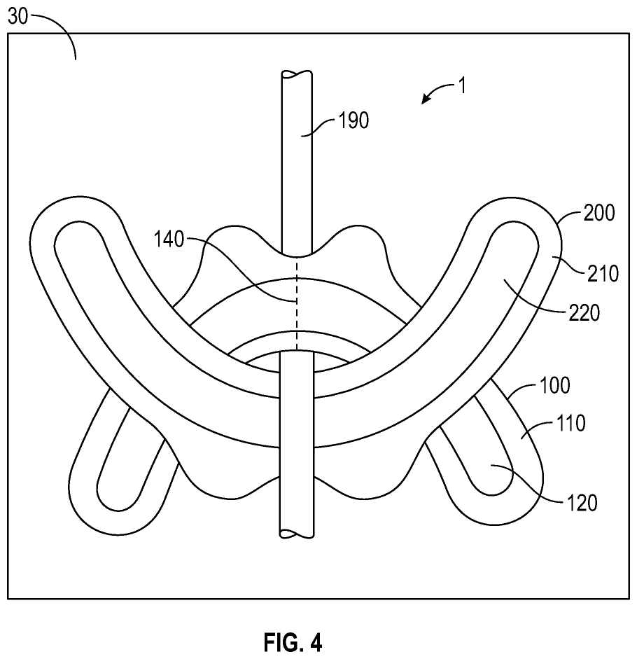

FIG. 4 shows the tape system 1 with the first tape 100 and second tape 200. Second tape 200 is substantially the same as first tape 100. Second tape 200 includes a midsection and a first leg and a second leg.

As shown in FIG. 4, first tape 100 is over tube 190 and overlies a substrate 30. Second tape 200 is under the tube 190, but with the first leg and the second leg of the second tape 200 placed over the first leg and the second leg of tape 100. The midsections of tapes 100 and 200 are aligned, but spaced apart, to allow, for example, tube 190 to be situated between the midsections of tapes 100 and 200. In this configuration, tape system 1 can advantageously secure tube 190 from being pulled away from the substrate.

In some embodiments, tapes 100 and 200 in tape system 1 are identical. A significant advantage of having tapes 100 and 200 be identical is simplification of applying the tape, in part by eliminating the need to select among two different types of tape during a catheterization process, where speed and ease of apply the tape system is highly valuable, to both the patient and the medical professional performing the catheterization. In some situations, only one type of the tape needs to be available to form the tape system, which can result in additional benefits in the areas of packaging, inventory, and cost.

In some other embodiments, tapes 100 and 200 in tape system 1 are not identical; for example, tapes 100 and 200 may differ from each other in shape, size, materials, in whether or not a perforation set is present, among other possible differences, or combinations of such differences. A tape system that uses non-identical tapes could be a useful, for example, in cases where the skin surface available for application of the tape system has restricted geometric requirements.

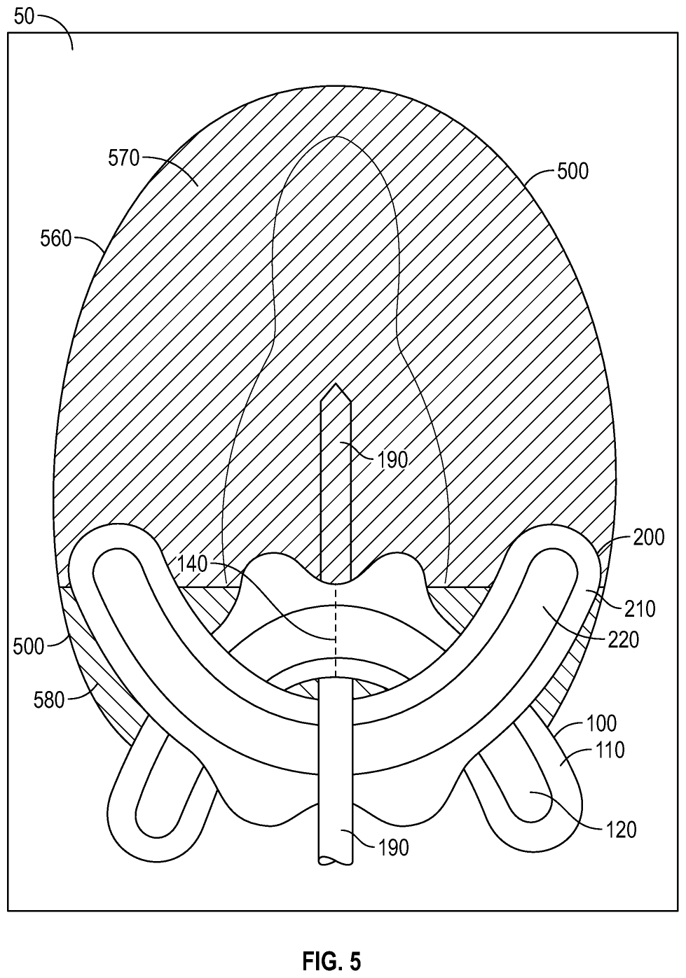

FIG. 5 shows a dressing system including the tape system 1 described above in FIG. 4 and an underlying dressing 500. Dressing 500 has a body layer 570 defined by a perimeter 560, body layer 570 having a first major surface opposite a second major surface. The second major surface has an adhesive. In the embodiment shown, a portion of body layer 570 includes a support layer 580. Dressing 500 overlies tube 190 and substrate 50. In embodiments where tube 190 is an IV catheter inserted into a vein, the dressing 500 can beneficially provide a physical protection of the insertion site from fluids, particulates, microorganisms, and the like. In the embodiment shown, a portion of tape system 1 is adhered to the first major surface of the dressing 500, and a portion of tape system 1 is adhered to substrate 50.

Backing Material

The backing layer 110 can be any material that provides mechanical stiffening of the tape while also allowing enough flexibility to allow for conformability and comfortable wear. Backing material can be a film, a paper, or a fabric layer material, such as a woven, knitted, or nonwoven fabric. Backing material can be elastic or stiff. In some embodiments, the backing layer material is a woven, knitted, or nonwoven material. One example of a nonwoven material is a high strength nonwoven fabric available from E. I. Dupont de Nemours & Company of Wilmington, Del., under the trademark SONTARA. Other suitable nonwoven webs include a hydroentangled polyester fabric available from Vertac, a division of International Paper of Walpole, Minn. Another suitable nonwoven web is the nonwoven elastomeric web described in U.S. Pat. No. 5,230,701. The backing can be a high moisture vapor permeable film backing. U.S. Pat. No. 3,645,835 describes methods of making such films and methods for testing their permeability.

The backing may be a single or multilayer construction. In some embodiments, an optional reinforcing layer material may be used with the backing. The reinforcing material may be as pliable as a thick adhesive or as stiff as a solid material (e.g., a paper or a film). For example, U.S. Pat. No. 5,088,483 discloses a permanent adhesive as a reinforcement material.

Adhesive

Any number of adhesives can be used with the tape and on the dressing, if included. Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber-based adhesives (including natural rubber, polyisoprene, polyisobutylene, butyl rubber etc.) as well as combinations of these adhesives. The adhesive component may contain tackifiers, plasticizers, rheology modifiers as well as active components, for example an antimicrobial agent.

The pressure sensitive adhesive is usually reasonably skin compatible and “hypoallergenic”, such as the acrylate copolymers described in U.S. Pat. No. RE 24,906. Particularly useful is a 97:3 iso-octyl acrylate:acrylamide copolymer, as is 70:15:15 isooctyl acrylate:ethyleneoxide acrylate:acrylic acid terpolymer described in U.S. Pat. No. 4,737,410. Additional useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Silicone adhesives can also be used. Generally, silicone adhesives can provide suitable adhesion to skin while gently removing from skin. Suitable silicone adhesives are disclosed in PCT Publications WO2010/056541 and WO2010/056543, the disclosures of which are herein incorporated by reference.

Adhesive layers of the present disclosure can be flood-coated, or pattern-coated. The adhesive layer is preferably pattern-coated to allow for better vapor transmission. An example of useful pattern coating is described in U.S. Pat. No. 4,595,001.

Dressing

In some embodiments, suitable dressings of the present disclosure include a thin, flexible and transparent polymeric film body layer. In some embodiments, the dressing further includes a support material. In general, the support layer materials can include, but are not limited to, an elastic film, a non-elastic film, non-woven fibrous web, woven fibrous web, knits, and polyethylene/vinyl acetate copolymer-coated papers and polyester films. Examples of suitable dressings can be found, for example, in WO2019/073326, U.S. Patent Application Publication No. US2016/0015570, and in U.S. Provisional Patent Application No. 62/827,400 “A Conformable Dressing” (filed Apr. 1, 2019). One example of a commercially available medical dressing is TEGADERM IV ADVANCED DRESSING (3M Co., St. Paul, Minn.).

Release Liner

In some embodiments, tapes and dressings of the present disclosure include a release liner film disposed on the skin adhesive layer (for example, adhesive layer 202 in FIG. 2). Release liners can be made of kraft papers, polyethylene, polypropylene, polyester or composites of any of these materials. The films are preferably coated with release agents such as fluorochemicals or silicones. For example, U.S. Pat. No. 4,472,480, the disclosure of which is hereby incorporated by reference, describes low surface energy perfluorochemical liners. The liners are papers, polyolefin films, or polyester films coated with silicone release materials. Examples of commercially available silicone coated release papers are POLYSLIK™, silicone release papers available from Rexam Release (Bedford Park, Ill.) and silicone release papers supplied by Loparex Group (Willowbrook, Ill.).

Kit

In some embodiments, a kit is provided that includes: 1) a dressing having a first major surface, a second major surface opposite the first major surface, defined by a perimeter around a body layer, the second major surface comprising a skin-compatible adhesive, with a first tape of the disclosure overlying the first major surface of the dressing; and 2) a second tape of the disclosure. In some embodiments, the entire first surface of the first tape overlies first major surface of the dressing.

In some embodiments, a portion of the body of the dressing includes a support layer, and the entire first surface of the first tape overlies the support layer.

The kit can facilitate applying the dressing and first tape together over a device inserted into a substrate, and applying the second tape under the device, with the first leg of the second tape applied over the second leg of the first tape, and with the second leg of the second tape applied over the first leg of the first tape, with the device extending between the midsection of the first tape and midsection of the second tape. In some embodiments, the first and second components of the kit can be disposed on a single release liner, to simplify application of the components to a patient.

Each embodiment shown in the figures is illustrated as a separate embodiment for clarity in illustrating a variety of features of the tape systems and dressing systems of the present disclosure. However, it should be understood that any combination of elements and features of any of the embodiments illustrated in the figures and described herein can be employed in the tape systems and dressing systems of the present disclosure.

1-21. (canceled)

22. A tape system comprising
a first tape comprising:
a first surface with a first tape adhesive and a second surface opposite the first surface;
a midsection extending in a longitudinal direction, the midsection having a first end and a second end;
a first leg extending from the midsection at the first end in a first lateral direction;
a second leg extending from the midsection at the second end in the first lateral direction; and
a second tape comprising:
a first surface with a second tape adhesive and a second surface opposite the first surface;
a midsection extending in a longitudinal direction, the midsection having a first end and a second end;
a first leg extending from the midsection at the first end in a first lateral direction;
a second leg extending from the midsection at the second end in the first lateral direction;
wherein the second tape is applied over the first tape with the first lateral direction of the first tape opposed to the first lateral direction of the second tape;
wherein the midsection of the first tape is aligned with but spaced apart from the midsection of the second tape; and
wherein at least a portion of the tape system overlies a first major surface of a dressing, wherein the dressing comprises the first major surface, a second major surface opposite the first major surface, defined by a perimeter around a body layer, the second major surface comprising a dressing adhesive.

23. The tape system of claim 22, wherein the longitudinal and first lateral directions in the first tape are perpendicular to each other.

24. The tape system of claim 22, wherein the midsection of the first tape extends in a straight line.

25. The tape system of claim 22, wherein the midsection of the first tape is curved.

26. The tape system of claim 22, wherein the first tape has a perforation set extending laterally across the midsection.

27. The tape system of claim 22, wherein the second tape is applied over the first tape with the first leg of the second tape applied over the second leg of the first tape, and with the second leg of the second tape applied over the first leg of the first tape.

28. The tape system of claim 22, wherein at least one of the first tape and second tape comprises a reinforcement layer.

29. The tape system of claim 22, wherein the first tape and the second tape are identical.

30. The tape system of claim 22, wherein a device extends between the midsection of the first tape and midsection of the second tape.

31. The tape system of claim 30, wherein the midsection of the first tape overlaps the device and the midsection of the second tape is under the device.

32. The tape system of claim 30, wherein the device is a tube.

33. The tape system of claim 22, wherein the entire first surface of the first tape overlies the first major surface of the dressing.

34. The tape system of claim 22, wherein the second major surface of the dressing overlies a substrate, and at least a portion of the tape system overlies the substrate.

35. A method of securing a device to a substrate, the method comprising:
applying the adhesive at the midsection of the first tape of claim 1 over a portion of the device with the first leg and second leg of the first tape adjacent to the device and overlying the substrate;
applying the midsection of the second tape of claim 1 under a portion of the device with the adhesive towards the substrate, wherein the first leg of the second tape overlies the second leg of the first tape, wherein the second leg of the second tape overlies the first leg of the first tape, and wherein the device extends between the midsection of the first tape and the midsection of the second tape.

36. The method of claim 35, further comprising applying a dressing over a portion of the device, the dressing comprising:
a first major surface, a second major surface opposite the first major surface, defined by a perimeter around a body layer, wherein the second major surface comprises an adhesive, and wherein the second major surface overlies the substrate.

37. The method of claim 36, wherein the dressing is applied over the portion of the device prior to the applications of the first and second tapes.

38. A kit comprising:
1) a dressing comprising:
a first major surface, a second major surface opposite the first major surface, defined by a perimeter around a body layer, the second major surface comprising a dressing adhesive;
2) a first tape comprising:
a first surface with a first tape adhesive and a second surface opposite the first surface;
a midsection extending in a longitudinal direction, the midsection having a first end and a second end;
a first leg extending from the midsection at the first end in a first lateral direction;
a second leg extending from the midsection at the second end in the first lateral direction;
3) a second tape comprising:
a first surface with a second tape adhesive and a second surface opposite the first surface;
a midsection extending in a longitudinal direction, the midsection having a first end and a second end;
a first leg extending from the midsection at the first end in a first lateral direction; and
a second leg extending from the midsection at the second end in the first lateral direction.
